(19) United States
(12) Patent Application Publication
(10) Pub. No.: US 2019/0333631 A1
(43) Pub. Date: Oct. 31, 2019

(54) CLOUD-BASED PORTAL FOR MONITORING BIOMETRICS

(71) Applicant: PMD Healthcare, Allentown, PA (US)

(72) Inventor: Wayne Meng, Fogelsville, PA (US)

(21) Appl. No.: 16/399,374

(22) Filed: Apr. 30, 2019

Related U.S. Application Data
(60) Provisional application No. 62/664,916, filed on Apr. 30, 2018.

(51) Int. Cl. G16H 40/20; G16H 15/00; G16H 80/00; A61B 5/00

(52) U.S. Cl. CPC G16H 40/20; A61B 5/0022; G16H 80/00; G16H 15/00

(57) ABSTRACT

A Wellness Management Services (WMS system) for use in a healthcare system in which a healthcare provider must spend a minimum unit of time on a single patient within a billing period to bill for said time, said WMS system comprising: (a) a provider portal configured to display a general page in which a plurality of patients are listed along with links to patient-specific pages, at least a portion of said patient-specific pages displaying biometric data, said provider portal also being configured to display said patient-specific pages upon selection by said provider; and (b) a computational module being configured to record session time said provider spends on each of said patient-specific pages in a given session, and being configured to calculate total time said provider spent on said patient-specific pages for all sessions within said billing period for each of said plurality of patients.

### RELATED APPLICATION

This application is based on U.S. Provisional Application No. 62/664,916, filed Apr. 30, 2018, and incorporated by reference herein in its entirety.

### FIELD OF INVENTION

This application relates generally to a portal to facilitate telemedicine, and, more specifically, to a portal configured to monitor and aggregate time a healthcare provider spends remotely monitoring a patient's biometric data or addressing other healthcare issues.

### BACKGROUND

Telemedicine is the use of telecommunication and information technology to provide clinical health care from a distance. It has been used to overcome distance barriers and to improve access to medical services that would often not be consistently available. Additionally, telemedicine allows one provider to monitor and interact with patients in a mere fraction of the time required for a face-to-face visit.

Ironically, the efficiencies afforded by telemedicine present challenges for healthcare providers to be adequately compensated for the time they spend providing non-face-to-face services. Specifically, many health care insurance systems have minimum times that can be billed. The time a provider spends providing non-face-to-face services via telemedicine for a single session is frequently below the minimum time requirements for billing.

One way to avoid this problem is to aggregate the time a provider spends on a patient within a billing cycle. This, however, presents its own challenges. It is administratively burdensome to record, with sufficient specificity, the time a provider may spend reviewing biometric data (i.e., Remote Patient Monitoring (RPM)) or providing other non-face-to-face services. Indeed, adequately recording the time spent may exceed the actual time spent in connection with the service. On the other hand, if a provider attempts to reduce the administrative burden by minimizing the specificity of the recorded time, then the time entries may not have the requisite detail for reimbursement, or otherwise provide an adequate audit trail.

Therefore, what is needed is a system for facilitating telemedicine by allowing the provider to quickly access important biometric data as well as other information while automatically recording and documenting the provider's time spent on reviewing and monitoring this data with minimum administrative burden. The present invention fulfills this need among others.

### SUMMARY OF INVENTION

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

The invention provides relates to a system and method for facilitating telemedicine by allowing the provider to quickly access important biometric data as well as other information while automatically recording and documenting the provider's time spent on reviewing and monitoring this data with minimum administrative burden. In one embodiment, the invention relates to a Wellness Management Services (WMS) system for use in a healthcare system in which a healthcare provider must spend a minimum unit of time on a single patient within a billing period to bill for the time, the WMS system comprising: (a) a provider portal configured to display a general page in which a plurality of patients are listed along with links to patient-specific pages, each patient-specific page corresponding to one of the plurality of patients, at least a portion of the patient-specific pages displaying biometric data, the provider portal also being configured to display the patient-specific pages upon selection by the provider; and (b) a computational module configured to record session time the provider spends on each of the patient-specific pages in a given session, and configured to calculate total time the provider spent on the patient-specific pages for all sessions within a given billing period for each of the plurality of patients.

In another embodiment, the invention relates to a Wellness Management Services (WMS) method for use in a healthcare system in which a healthcare provider must spend a minimum unit of time on a single patient within a billing period to bill for the time, the WMS method comprising: (a) displaying a general page in which a plurality of patients are listed along with links to patient-specific pages, each patient-specific page corresponding to one of the plurality of patients, at least a portion of the patient-specific pages displaying biometric data, the provider portal also being configured to display the patient-specific pages upon selection by the provider; (b) automatically recording session time the provider spends on each of the patient-specific pages in a given session; (c) automatically calculating total time the provider spends on the patient-specific pages for all sessions within the billing period for each of the plurality of patients; and (d) providing a record of the total time in a form sufficient to meet health-care documentation requirements for insurance reimbursement.

Therefore, in one embodiment, the total time the provider spends on the patient-specific pages for all sessions within the billing period for each of the plurality of patients is calculated. As mentioned above, it is not unusual for a session time to be below the minimum billable unit, yet the total time to be above the minimum. Thus, by totaling these session times within a given billing period, the minimum billable unit of time is more likely reached, and thus the provider is able to bill his or her time. Moreover, the total time is presented in a form sufficient to meet health-care documentation requirements for insurance reimbursement (e.g., auditable). (Such requirements are well known to those knowledgeable in health-care reimbursement.)

### BRIEF DESCRIPTION OF FIGURES

FIG. 1 shows a screenshot of one embodiment of the primary page.

FIG. 2 shows a screenshot of the activity summary (or non-face-to-face time summary)

FIG. 3 shows a screenshot of a non-face-to-face time summary for a particular patient.

FIG. 4 shows a screenshot of a patient education summary for a particular patient.

FIG. 5 shows a screenshot of the video/phone conferencing page.

FIG. 6 shows a screenshot of an activity time logging page.

FIG. 7 shows the screenshot of FIG. 6 with a patient selected.

FIG. 8 shows a screenshot of a provider summary.

FIG. 9 shows a screenshot of a patient data entry page.

FIGS. 10(a) through 10(d) show screenshots of secondary pages regarding biometric trend data.

FIG. 11 shows a screenshot of a prompt from the system to determine whether the provider is still viewing a secondary page.

FIG. 12 shows a screenshot of a non-face-to-face timer once the start timer button is clicked on the activity time logging page shown in FIG. 7

FIG. 13 shows a screenshot of a time entry page for entering time as an alternative to using a timer.

FIG. 14 shows a screenshot of a portal registration page.

FIG. 15 shows a screenshot of one embodiment of the patient portal general page.

### DETAILED DESCRIPTION

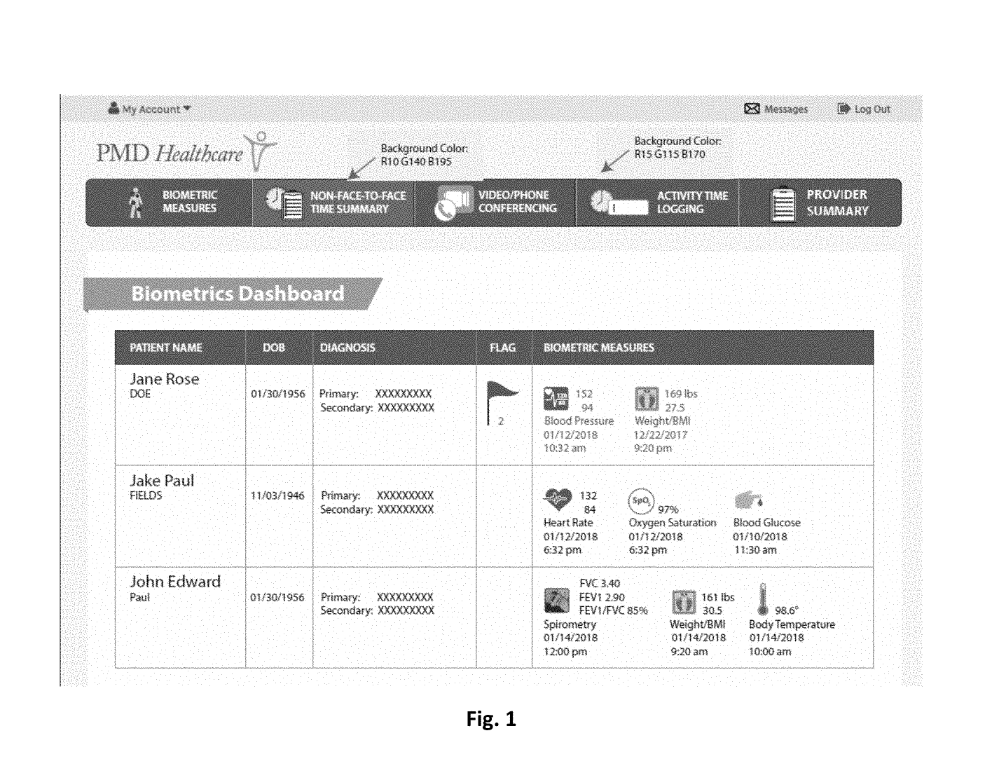

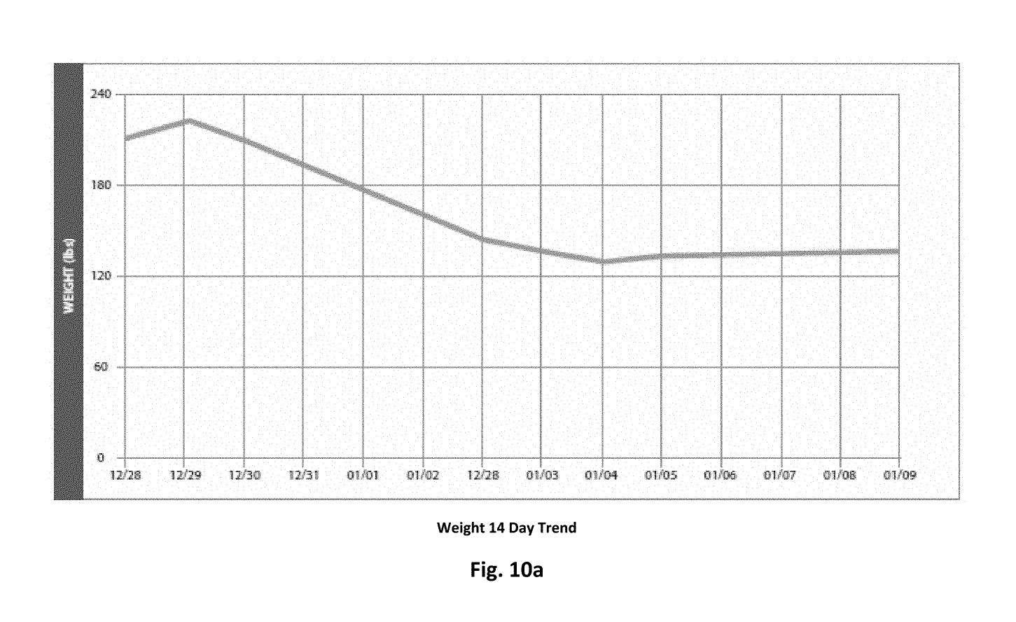

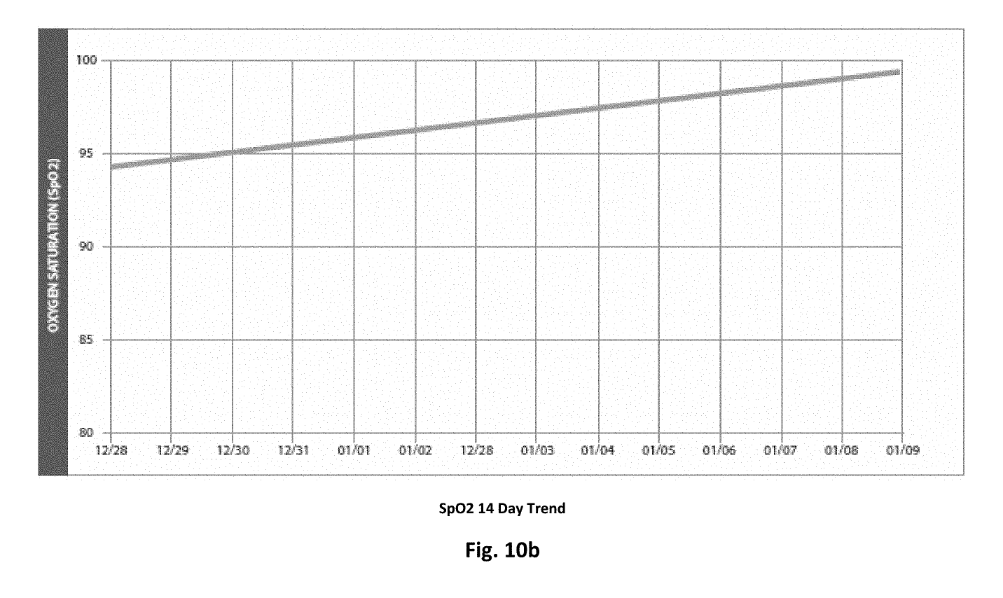

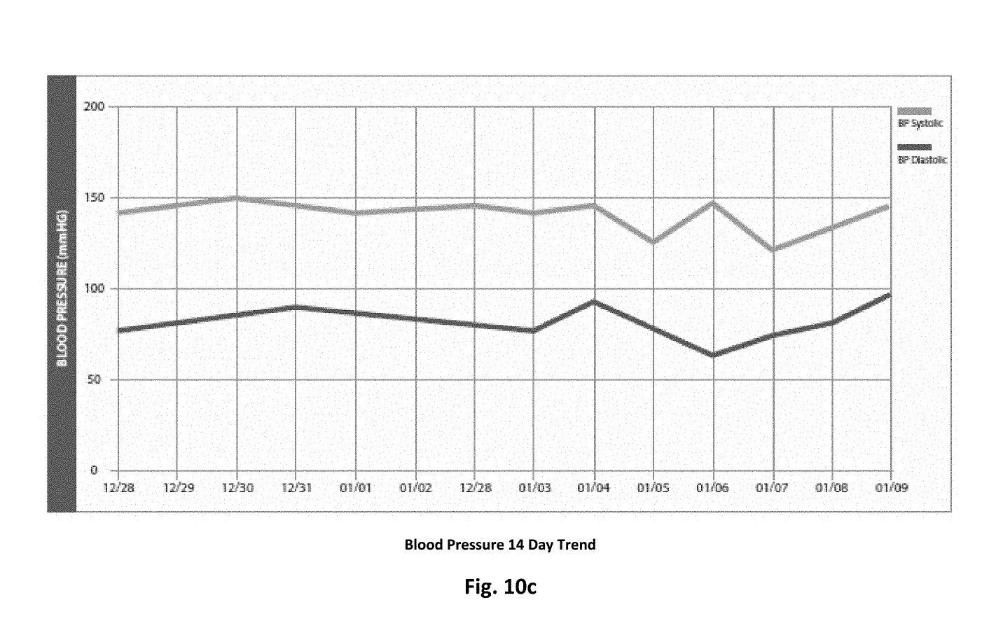

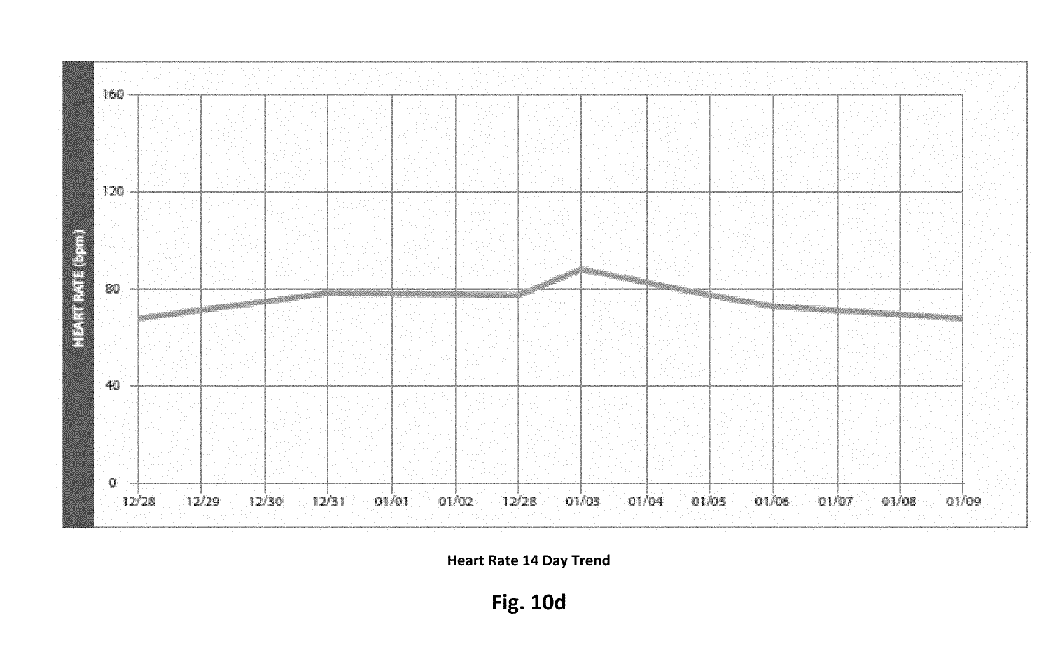

Referring to FIGS. 1, 2, and 10(a)-(d), one embodiment of the Wellness Management Services (WMS) system of the present invention is shown. The WMS system is particularly well-suited for use in a healthcare system in which a healthcare provider must spend a minimum unit of time on a single patient (e.g., ½ hr.) within a billing period to bill for the time. In this embodiment, the WMS system comprises a provider portal configured to display a general page (FIG. 1) in which a plurality of patients is listed along with links to patient-specific pages (see e.g., FIGS. 10(a)-10(d)), each patient-specific page corresponding to one of the plurality of patients, at least a portion of the patient-specific pages displaying biometric data. The provider portal is configured to display the patient-specific pages upon selection by the provider. The WMS system also comprises a computational module configured to record session time the provider spends on each of the patient-specific pages in a given session. The computational module is also configured to calculate total time the provider spent on the patient-specific pages for all sessions within a given billing period for each of the plurality of patients. These features are described below in greater detail, along with selected alternative embodiments.

General Page

Referring to FIG. 1, one embodiment of the general page is shown. The general page in this embodiment displays a biometric dashboard for a plurality of patients. This page contains a row for each patient assigned to the logged-in Provider. In this embodiment, the top button bar contains the following buttons:
Biometric Measures: this is the default page displayed, and when this button is selected, the Biometrics Dashboard may be displayed.
Non-Face-to-Face Time Summary: when this button is selected, the Non-Face-To-Face Time Summary Dashboard may be displayed.
Video/Phone Conferencing: when this button is selected, the Video/Phone Conferencing page may be displayed.
Activity Time Logging: when this button is selected, the Non-Face-to-Face Activity

Time Logging page may be displayed
Provider Summary: when this button is selected, the Provider Summary Page may be displayed

Time Keeping

It should be understood that an important feature of the system is the ability to total the time the provider spends monitoring biometrics, non-face-to-face conferencing with the patient, and otherwise rendering a non-face-to-face patient service, over an entire billing period for a given patient. Accordingly, in one embodiment, the system comprises a computational module for performing this function. In one embodiment, the computational module is configured to calculate the total time by totaling all time spent on the patient-specific pages for a given patient for all sessions within a given billing period. In this embodiment, the total is calculated for given period for non-face-to-face healthcare services. Alternatively, in another embodiment, the computational module is configured to calculate the total time spent on patient-specific pages by itemizing time spent on each of the patient-specific pages for a given patient for all sessions within a given billing period. Generally, although not necessarily, these totals are calculated by totaling all the session times for a given billing period. As mentioned above, it is not unusual for a session time to be below the minimum billable unit, but the total time is above the minimum. Thus, by totaling these session times within a given billing period, the minimum billable unit of time is more likely reached, and thus the provider is able to bill his or her time.

It is important that the computational module provides an auditable account of time spent on a particular patient. Therefore, in one embodiment, the computational module comprises a timer for calculating the session time. In one embodiment, the system automatically calculates the session time the provider is on a patient-specific page and provides a record of that session for audit purposes.

In one embodiment, the system is configured to validate that the provider is indeed engaged actively WMS system. Although the system may be configured in different ways to do this, in one embodiment, the computational model comprises/is associated with an activity monitor to ensure that the provider is actively viewing and interacting with the patient-specific page. For example, in one embodiment, the activity module is configured to monitor provider activity while on a patient-specific page (e.g. keyboard clicks, mouse movement, etc.), and, after a predetermined first period of detecting no provider activity while on the patient-specific page, to prompt the provider to determine if the provider is still viewing the patient-specific page. In one embodiment, if the provider does not respond affirmatively, then the timer is stopped. Further, in one particular embodiment, a predetermined second period is subtracted from the session time if the activity module determines that the provider is not actively engaged with the patient—specific page. For example, in one embodiment, if the provider affirmatively indicates that he/she is not actively engaged with the patient—specific page, then a period, for example, a period equal to the duration of inactivity plus the duration the prompt was displayed before receiving a response from the provider, may be subtracted from the session time. If the provider fails to respond to the prompt, then an even longer period may be subtracted from the session time. The predetermined first and second times in this embodiment may vary according to compliance requirements. For example, in one embodiment, the predetermined second period is at least equal to the predetermined first period to ensure that the time the provider spends on the patient-specific page is indeed active time, warranting reimbursement. In this respect, all of the parameters of the computational module may vary according to the application and reimbursement compliance requirements. Below is an example of one embodiment.

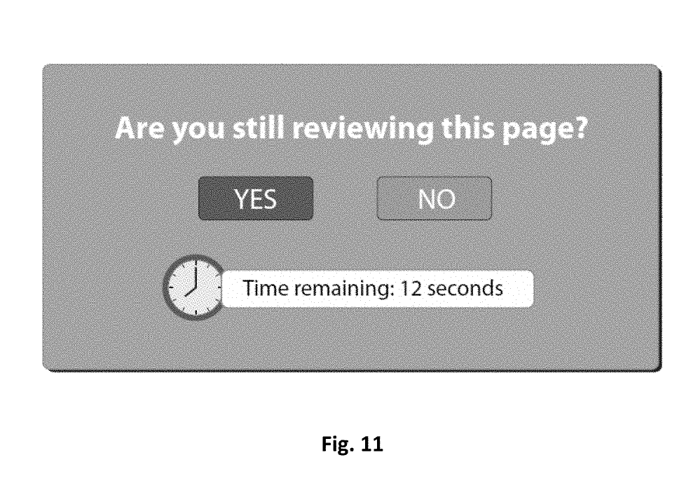

In one embodiment, whenever the provider views any patient-specific page for a patient, the system time calculations follow these rules:
Any page view time less than 1 second is considered not viewed and not be logged. (Thus, in this embodiment, the system requires a minimum time be spent on a webpage otherwise the page is not considered viewed.)
Each patient's portal page viewed by the provider is logged with a date/time stamp, number of seconds viewed, and who viewed it.
If on any page there is no user (provider) activity, including mouse moves, mouse clicks or keyboard actions for 30 seconds (i.e., the first predetermined period), then the following shall occur:
   a. A pop-up may be displayed with a sound containing the text “Are you still reviewing this page?” with a “Yes” button and a” No” button. This pop-up displays a countdown field, starting at 15 seconds, and shall decrement every second. This pop-up is shown in FIG. 11.
   b. If the provider selects “Yes”, then the pop-up is removed and the internal second counting shall continue normally.
   c. If the provider selects “No”, then the pop-up is removed and 30 seconds (i.e., the second predetermined time) is subtracted from the total number of seconds provider viewed this page.
   d. If the countdown decrements down to 0 seconds, the pop-up may be removed, 45 seconds may be subtracted from the total number of seconds the provider viewed this page and the page shall display the provider's biometric dashboard.

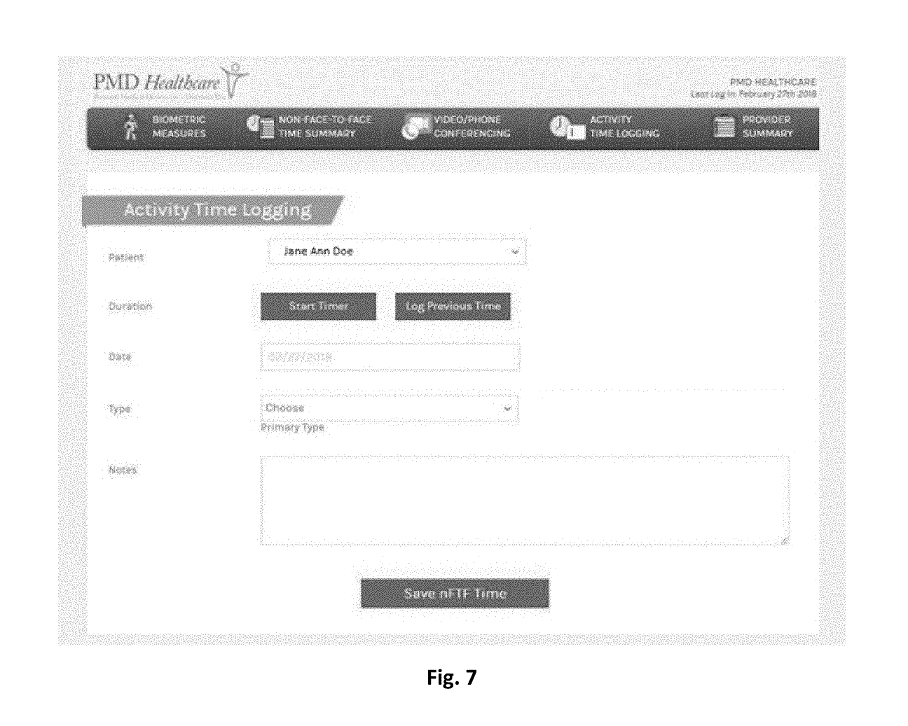

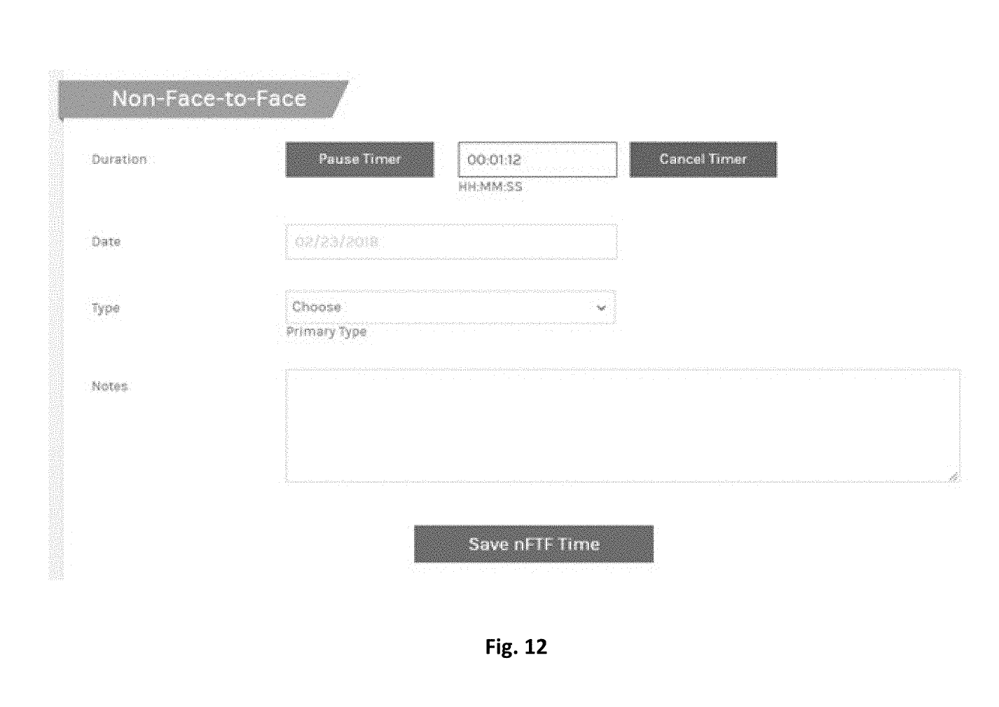

In an alternate or additional embodiment, time recording can be achieved through the activity time logging page. Referring to FIG. 7, one embodiment of this page is depicted. In this embodiment, there is a “Choose Patient” dropdown displayed. In one embodiment, until a patient is selected, all of the remaining controls on this page, specifically “Start Timer” button, “Log Previous Time” button, “Date” Calendar, “General” Type dropdown, “Notes” field, and the “Save nFTF Time” button remain disabled. Once a patient is selected, these disabled controls are enabled and the page shown in FIG. 12 below may be displayed.

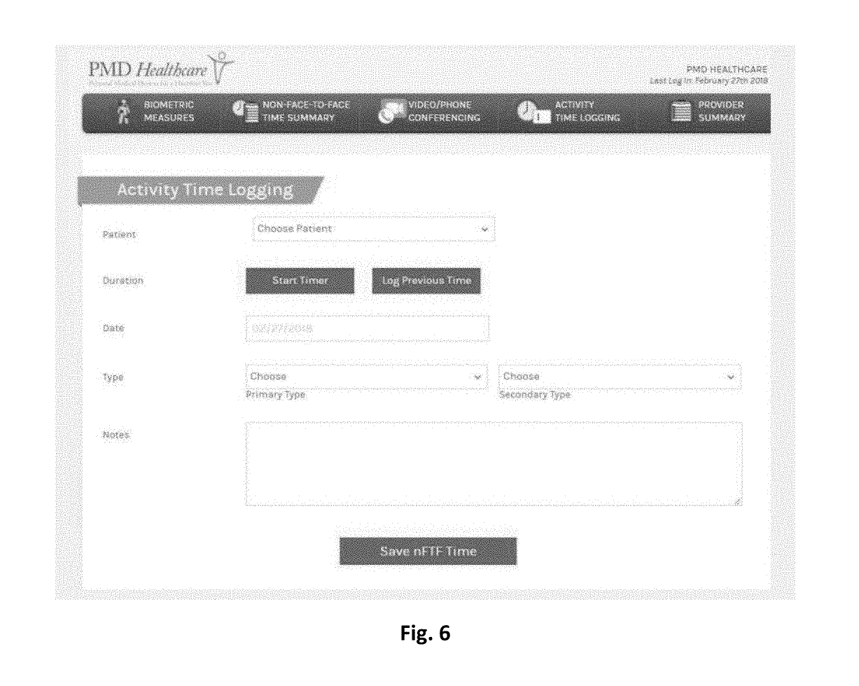

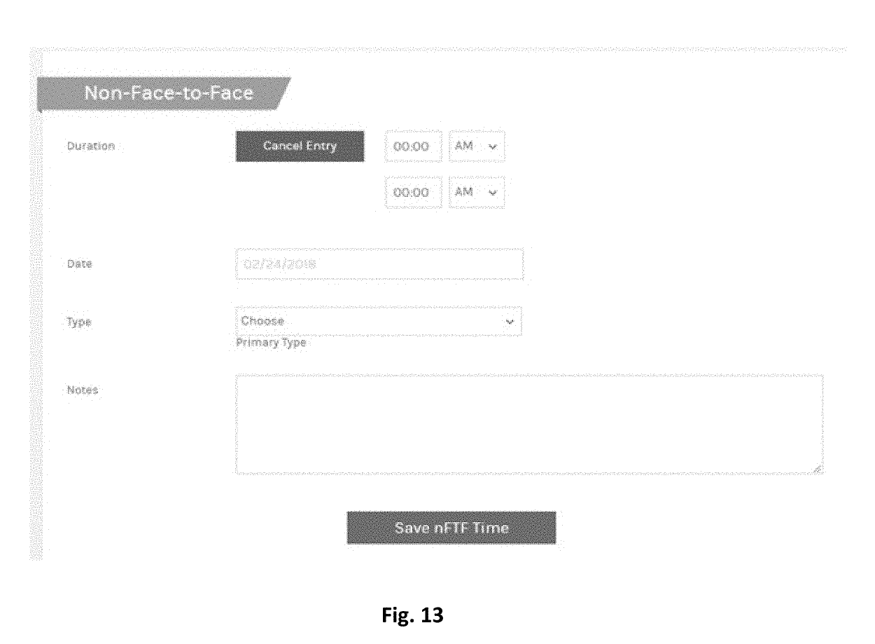

Referring to FIG. 12, once the Activity Logging page is displayed, the following actions may be supported:
If the provider presses the Start Timer button as shown for example in the screenshot of FIG. 7, the screenshot of FIG. 12 may be displayed showing a timer incrementing every second and a red Cancel Timer button. Note, in this embodiment, a Primary type selection is not necessary for the timer to start, although, in other embodiments it might be.
If the Pause Timer button is selected, the timer shall pause and this button shall change to “Resume Timer”.
If the Resume Timer button is selected, the timer shall continue to increment every second.
If the Cancel Timer button is selected, the timer shall stop and the page shall revert back to the Activity Timer page in FIG. 6 above.
   If the user selects the Log Previous Time button as shown in the page of FIG. 6, the page as shown in FIG. 13 may be displayed allowing the user to enter a start and end time for any date previously done.
   In one embodiment, if the Provider presses the Cancel Entry button in the page of FIG. 13, the Log Previous Time page is removed.
   The Date field may be automatically filled by default with the current date. In one embodiment, if the provider selects the Date filed, a calendar shall appear allowing the provider to select a date from the calendar.
   In one embodiment, the Type drop down lists shall contain a Primary type dropdown list. The contents of the Primary type dropdown list may include, for example:
      a. Phone Time
      b. Education
      c. Review Patient Information
      d. Care Coordination
      e. Medication Change
      f. Medication Review/Reconciliation
      g. Other
   The Notes field may be a free text entry field that may be included, if entered, with the saved entry.
   If the user selects the Save nFTF Time button, a check may be made to make sure the date, a start time with duration (or for previous time and End Time), and a Primary type are chosen. In one embodiment, if all of these criteria are met, the entry may be saved. If any of the check criteria are not met, an error message shall appear for the user to correct.

Considering next are patient-specific pages and non-face-to-face services provided to the patient by the provider.

Monitoring Biometrics

Now the functionality of each of the tabs in the general page will be considered. Considering biometric measures first, in one embodiment, the biometrics dashboard is the default general page. Referring to FIG. 1, the following columns are displayed in the biometrics dashboard/general page:
Patient Name: this may be a sortable column and the sorting occurs on the Last Name. For each patient there may be a First Name, Middle Name and a Last Name displayed. Fig.
DOB: Date of Birth, this may be a sortable column. The patient's DOB may be displayed in the following format: mm/dd/yyyy, where month and day are 0-filled (01, 02, etc.)
Diagnosis: this may be a sortable column and the sorting occurs on the General Diagnosis. The General diagnosis may be displayed for each patient. If the patient has a Patient-specific diagnosis, the Patient-specific diagnosis may also be displayed.
Flag: this may be a sortable field and the sorting occurs on the number of active flags (described below) for the patient. There may be either no flag or one flag displayed depending on the flag rules specified below. The display of the flag may include a number showing the number of flags active.
Biometric Measures—this is not typically a sortable column. The data contained may be an icon for each biometric device the patient has plus the name and date/timestamp of the last reading. The display order of these biometrics may be based on the date/timestamp of each reading with the most recent reading displayed in the left most position. In one embodiment, up to three biometric icons may be displayed in a row, and, if there are more than three, then a new row may be displayed to accommodate more than three icons. In one embodiment, if a biometric value is outside the normal range for that biometric (list and values of these ranges will be provided), that value and date/timestamp, title or icon may be emphasized in some way to call attention to it. For example, in one embodiment, the data is displayed in red.

In one embodiment, the order in which the patients are listed may depend upon their biometric data. For example, when the Biometrics Dashboard is initially displayed, the patient sort order may be according to the following rules:
Flag set, patient with most flags on top
Highlighted biometric (in red), patient with highest number of highlighted biometric data on top
Order of patients with same issue:
   a. Blood Pressure (top-most)
   b. Heart Rate
   c. Weight/BMI
   d. SpO2
   e. Spirometry
Sort by Patient's Last Name

The display of a flag for a patient may occur under various circumstances. For example, in one embodiment, a flag occurs based on an alert in a biometric measurement exceeds a predetermined limit or baseline, which, in one embodiment, is defined in the portal and include the following:
FVC alert
FEV1 alert
FEV1/FVC alert
ACT alert
CAT alert
Weight alert
Blood Pressure alert
Heart Rate alert
SpO2 alert
Blood Glucose alert
Body Temperature alert

It should be understood that exceeding a predetermined limit is not limited to a biometric measure exceeding an upper limit, but also means that the biometric measure goes beyond a lower limit. In other words, exceeding a predetermined limit means that the biometric measure is outside of an accepted range.

As mentioned above, from the general page, the provider may select a patient-specific page displaying additional biometric data. The provider may navigate to a patient-specific page in different ways. For example, in one embodiment, if the Provider clicks on the patient's name, that patient's dashboard page may be displayed allowing the Provider to review the patient's data. In another embodiment, if the Provider hovers the mouse pointer over a patient's Biometric Measurement data, additional biometric data, for example, a trend graph, as shown in FIGS. 10(a)-(d), may be displayed as a window overlay on the dashboard. In yet another embodiment, if the Provider clicks on a biometric for a patient, a patient-specific page providing additional biometric data for that patient as shown in FIGS. 10(a)-(d), may be displayed.

For example, FIGS. 10a through 10d show trend data. Briefly, FIG. 10a shows a weight trend over a 14-day period, FIG. 10b shows an oxygen saturation trend for a 14-day period, FIG. 10c shows a blood pressure trend for a 14-day period, and FIG. 10d shows a heart rate trend for a 14-day period. One of skill in the art will understand that the duration of the trends can be changed according to provider/patient preference.

Although trend data is illustrated in this example, it should be understood that other biometric data may also be displayed in patient-specific pages. For example, biometric data may be displayed in tabular form over an extended period, greater resolution of the data can be provided, or the data may be compared to norms.

Video/Phone Conferencing

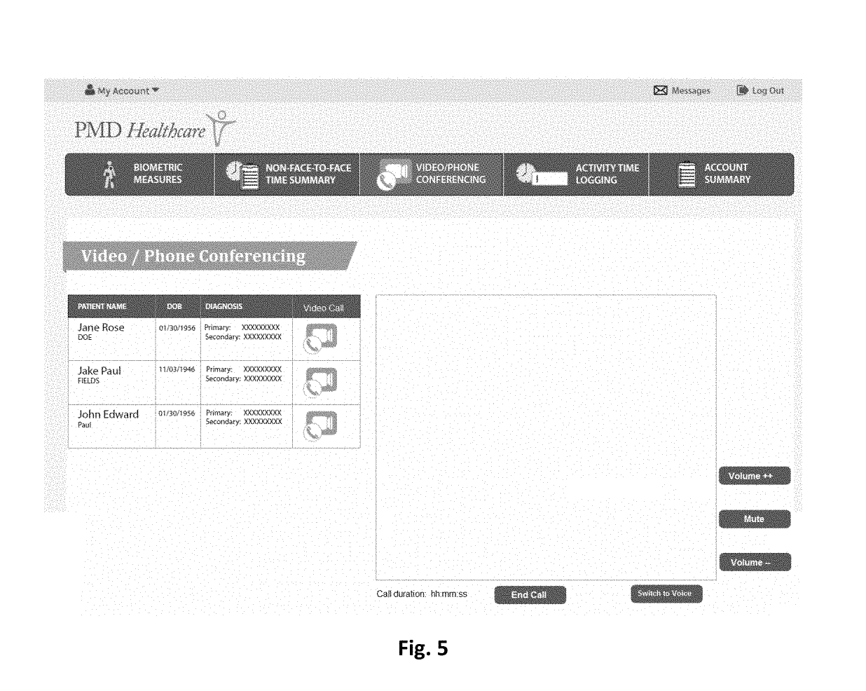

In addition to monitoring biometric data, telemedicine also provides for other non-face-to-face services, which need to be recorded as well for reimbursement purposes. For example, in one embodiment, the system facilitates video/phone conferencing by providing a video/phone conferencing page as shown in FIG. 5. In one embodiment, the video call is logged for a particular patient including date and time of the call and the duration. In this particular embodiment, the display has the following features:
List of all patients associated for this provider, including name, date of birth (DOB) and Diagnosis.
The columns for Name, DOB and Diagnosis may be sortable.
The last column shall contain a video call icon. If the provider selects this icon, the corresponding patient is called and the Video Call section is enabled.
A Video Call section on the right side of the page.

In one embodiment, the Video Call section shall contain the following:
Viewable video section for seeing the patient.
Display of call duration updated every second.
Button for Mute/Unmute volume
Button for Increase Volume
Button for Decrease Volume
Button for Switch to Voice (toggles to Switch to Video)
Button for End Call

In one embodiment, the Video Call Section is disabled until the provider makes a Video Call as described above, where all buttons may be grey and non-functional while the “Call duration:” text shall also be grey.

Monitoring Patient Medical Compliance

In one embodiment, the system provides for monitoring a patient's medical compliance. Lack of medical compliance is recognized as a contributing factor in prolonging or exasperating illnesses. As part of a non-face-to-face service, the provider accesses a patient that specific-page to monitor the patient's medical compliance and to determine whether corrective action is required.

Monitoring Patient Education

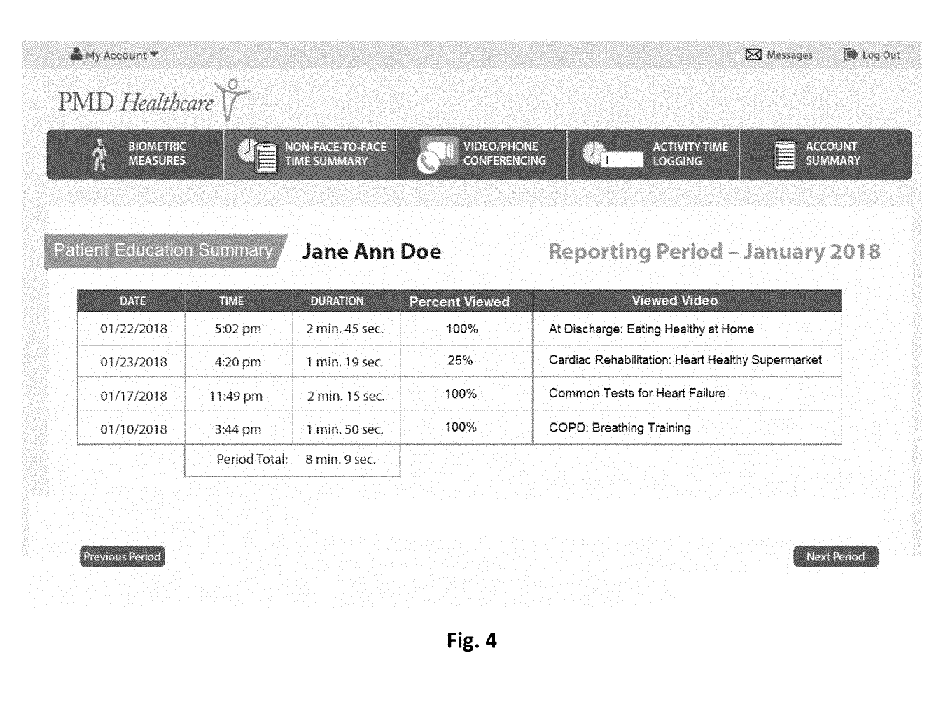

In one embodiment, the system provides for patient education. In such an embodiment, it may be preferable for the provider to monitor the patient's progress in reviewing the educational materials. Accordingly, in such an embodiment, a Patient Education Summary Page is provided as shown in FIG. 4.

Non-Face-to-Face Summary

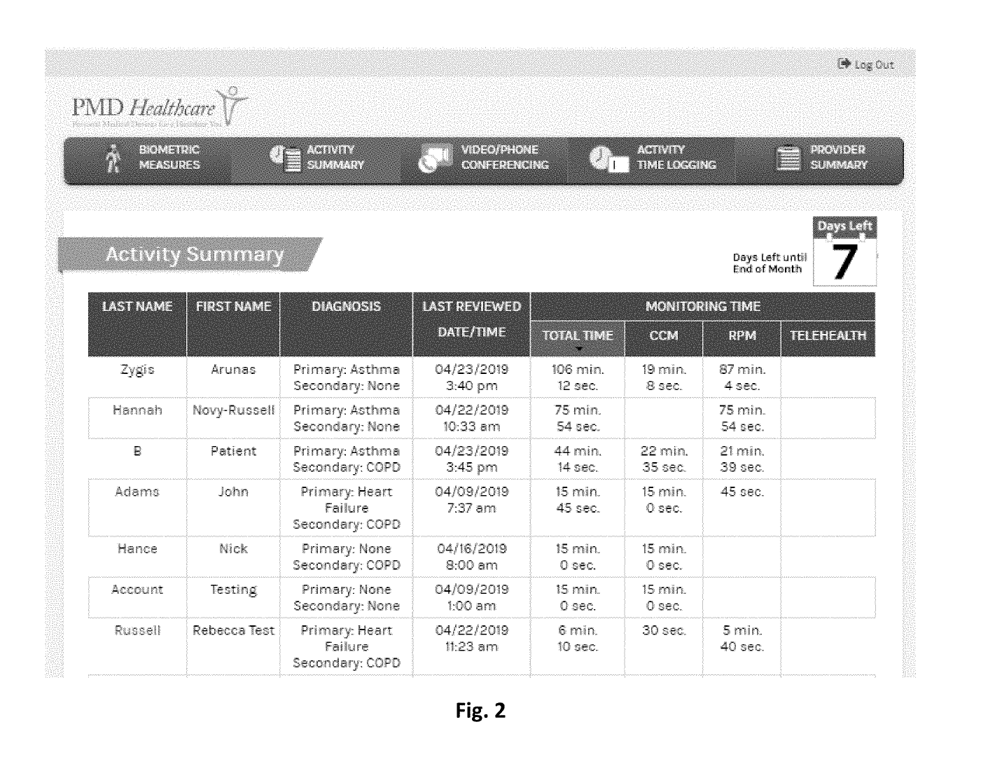

Referring to FIG. 2, a screenshot of one embodiment of a non-face-to-face time summary dashboard is shown. In this embodiment, the page displays a row for each patient assigned to the logged-in Provider. Below are examples of the columns that may be displayed:
Patient Name—this may be a sortable column and the sorting occurs on the Last Name. For each patient there may be a First Name, Middle Name and a Last Name displayed.
Diagnosis—this may be a sortable column and the sorting occurs on the General Diagnosis. The General diagnosis may be displayed for each patient. If the patient has a Patient-specific diagnosis, the Patient-specific diagnosis shall also be displayed.
Last Reviewed Date/Time—this may be a sortable column. The date and timestamp when this patient's record was last reviewed may be displayed here.
Monitoring Time—Total Time—this may be a sortable column. The Total Time monitored, which is the sum of Remote Patient Monitoring (RPM) and Conference, may be displayed here for each patient.
Monitoring Time—Remote Patient Monitoring (RPM)—this may be a sortable column. This Data Review displays the amount of time a Provider spends reviewing the patient's data since the start of the monitoring period. This time is automatically calculated by the portal software as the Provider goes into the patient's portal pages and reviews the data.
Monitoring Time—Telehealth Monitoring—this may be a sortable column. This Conference time displays the amount of time a Provider spends in a video/phone conference with this patient since the start of the start of the monitoring period.
Monitoring Time—CCM Monitoring—this may be a sortable column. This Activity time displays the Provider's Activity Time logged for this patient since the start of the start of the monitoring period.
Next Action—this is typically not a sortable column. This allows the Provider to (1) connect immediately to this patient's portal if the Portal icon is selected, (2) View the Patient Education Summary page if the Review Patient Education icon is selected.

In one embodiment, when the Non-Face-To-Face Time Summary Dashboard is initially displayed, the patient sort order may be according to the following rules:
Monitoring Total Time, with the lowest number on top
Sort by Patient's Last Name

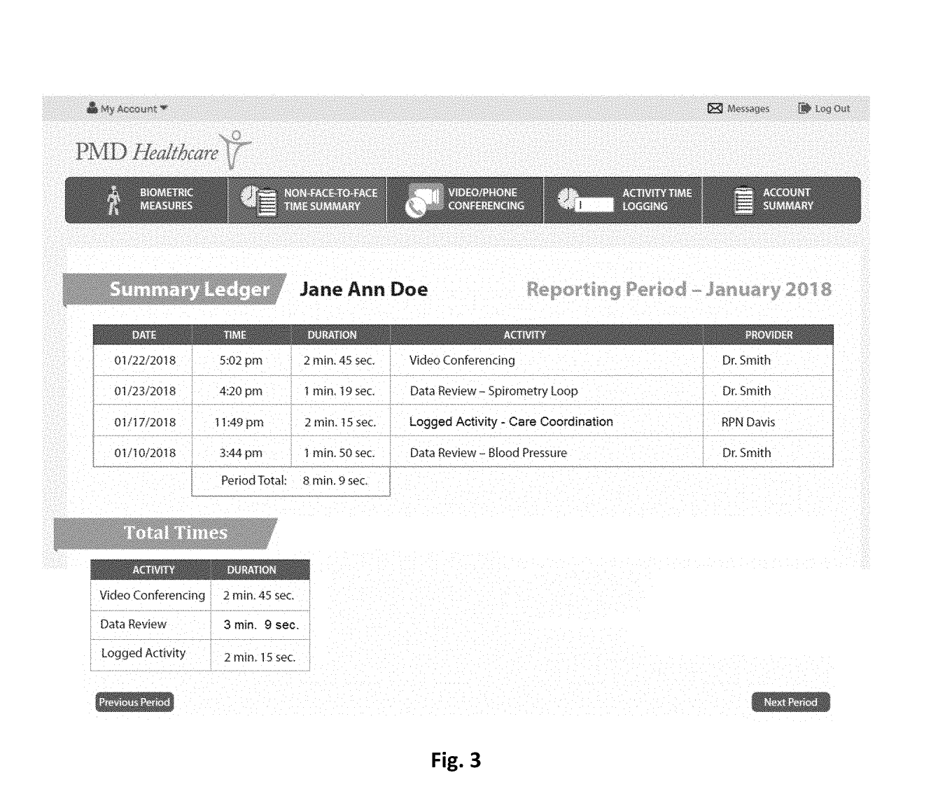

In one embodiment, if the Provider clicks on the patient's name, that patient's Summary Ledger page may be displayed allowing the Provider to review the patient's ledger as shown in FIG. 3.

The list below is an example of Activities in the Patient Summary Ledger. These billable activities are associated with a patient-specific page. In this particular embodiment, each “Data Review” entry represents a page in the patient's portal being viewed.
Video/Phone Conferencing
Data Review—Patient Biometrics Dashboard
Data Review—Spirometry All
Data Review—Spirometry FVC
Data Review—Spirometry FEV1
Data Review—Spirometry FEV1 / FVC
Data Review—Spirometry PEF
Data Review—Spirometry Loops
Data Review—Weight / BMI
Data Review—SpO2
Data Review—Heart Rate
Data Review—Blood Pressure
Data Review—Blood Glucose
Data Review—Body Temperature
Data Review—Diary View Calendar
Data Review—Diary ACT
Data Review—Diary CAT
Data Review—Diary Rescue Inhaler Usage
Data Review—Medication History
Data Review—Current Medication
Data Review—Tri-Trends
Data Review—Messages
Data Review—Biometric Alerts
Data Review—Therapy Alarms
Data Review—Medication Notifications
Data Review—Patient's Profile
Data Review—Device Settings
Logged Activity—Phone Time
Logged Activity—Education
Logged Activity—Review Patient Information
Logged Activity—Care Coordination
Logged Activity—Medical Change
Logged Activity—Medical Review/Reconciliation
Logged Activity—Other

Provider Summary Page

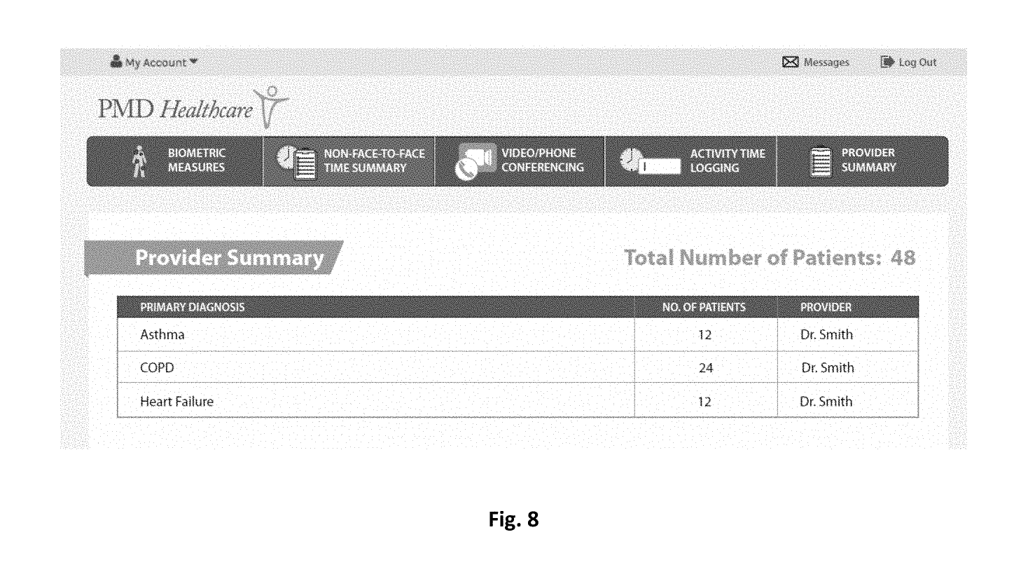

Referring to FIG. 8, one embodiment of the Provider Summary Page is shown. In this embodiment, the “Total Number of Patients” is the number of patients currently assigned to this provider. There may be a row in the table below for each general diagnosis for all the provider's patients. There may be a column showing the number of patients having each general diagnosis. There may also be a provider column showing the current provider name. Each column in the table may be sortable, with the default page view being sorted by General Diagnosis.

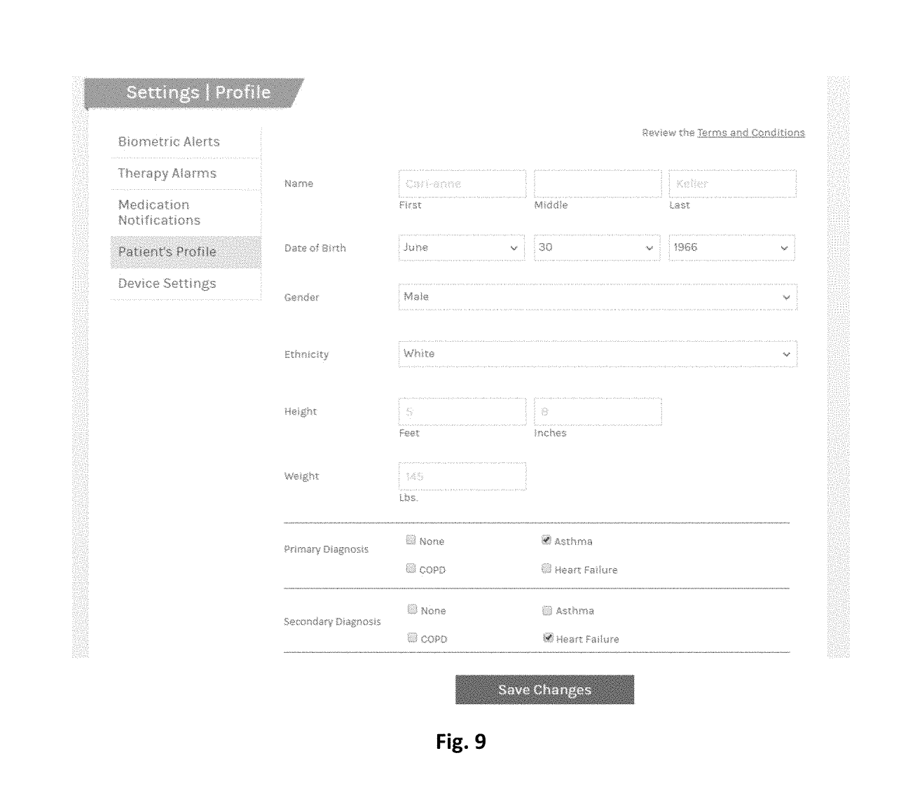

In one embodiment, the Chronic Conditions part of the Patient Profile page is configured to accommodate Primary and Secondary Diagnoses as shown in FIG. 9. In this embodiment, the default for the selections for both Primary and Secondary Diagnoses. Generally, the system allows one and only one selection for the Primary Diagnosis. If the Primary Diagnosis is “None”, the Secondary Diagnosis shall disable the non-“None” choices. If a Primary Diagnosis is selected, the user is able to select 1 or 2 non-None selections for the Secondary Diagnosis. The selection of the same Primary Diagnosis as the Secondary Diagnosis typically is not be allowed by the system.

Patient Portal

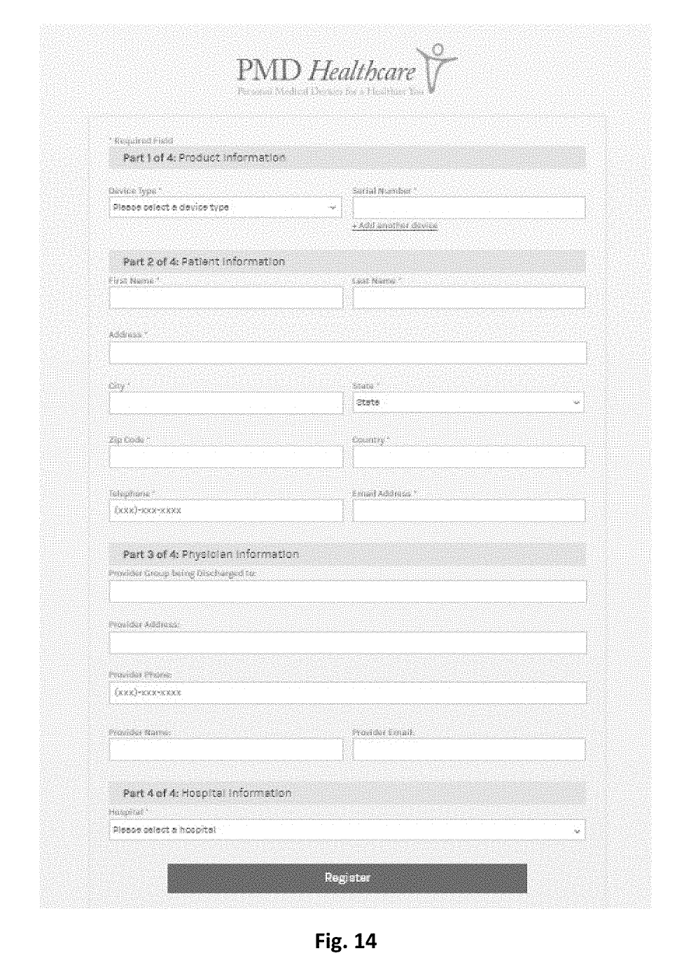

The patient portal, in one embodiment, serves several functions including, for example, entering biometric data. To this end, the patient portal must be configured to receive information from the patient through a centralized computer. In one embodiment, this information is received autonomously. Referring to FIG. 14, one embodiment of the screen for patient computer registration is shown. In this embodiment, when the patient registers his or her computer or tablet with the portal, there is a Device Type of “Tablet” available in the dropdown for “Device Type”. It should be understood that while this application refers generally to computers, the computer can be a number of different devices including, for example, a desktop computer, a laptop computer, a tablet computer, smart phone, and even a specially configured medical device, such as, for example a spirometer such as those available through PMD Healthcare.

Referring back to FIG. 14, the Hospital information, which is Part 4, is optional if the patient is registering a Tablet. In one embodiment, once the patient successfully registers his or her device, the patient receives an email which has a link available for the patient to enter a password. Once the patient completes this process on a separate browser device, that user id and password is recognized by the portal for subsequent logins. In another embodiment, for tablet users, PIN numbers are used for patients to login once and only one time while physically holding on to the tablet. Registration is handled through the admin panel

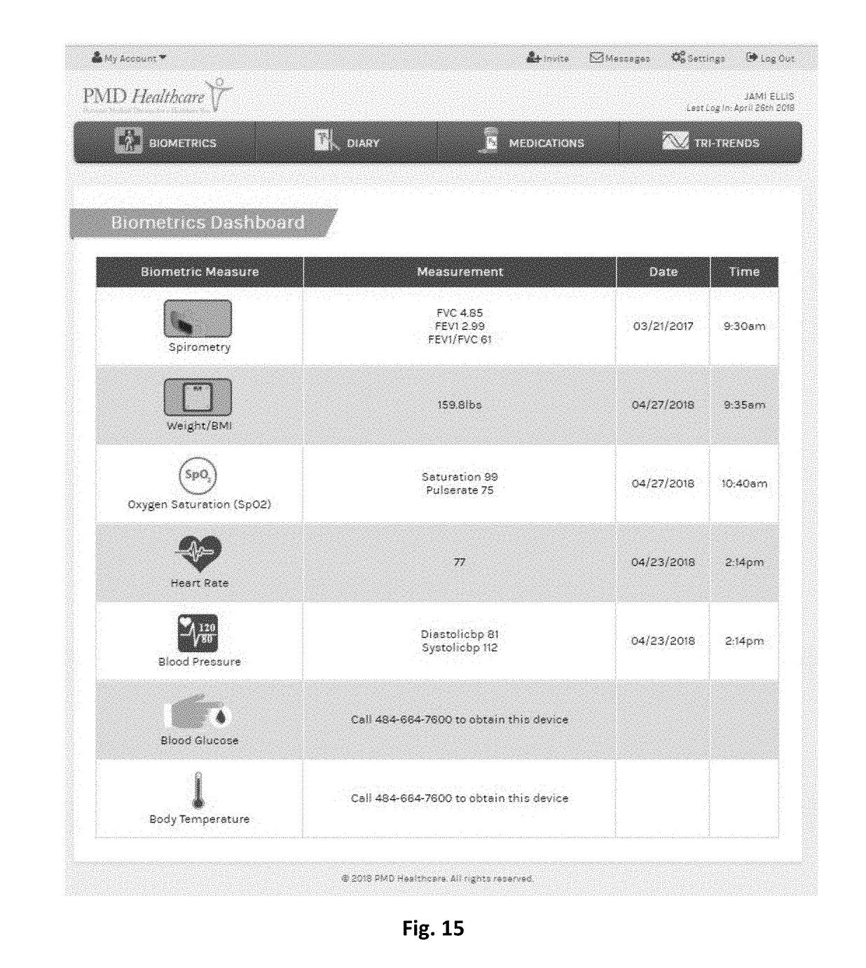

Referring to FIG. 15, a screenshot for one embodiment of the general page for the patient portal is shown. As with the provider portal, the default page, in this embodiment, is a biometrics dashboard. At the top of this page, are a number of tabs corresponded to biometrics, diary, medications, and tri-trends. Each one of these tabs provides the patient with useful information concerning their treatment, medical compliance, and performance.

It should be understood that the foregoing is illustrative and not limiting and that obvious modifications may be made by those skilled in the art without departing from the spirit of the invention. Accordingly, the specification is intended to cover such alternatives, modifications, and equivalence as may be included within the spirit and scope of the invention as defined in the following claims.

What is claimed is:

1. A Wellness Management Services (WMS system) for use in a healthcare system in which a healthcare provider must spend a minimum unit of time on a single patient within a billing period to bill for said time, said WMS system comprising:
a provider portal configured to display a general page in which a plurality of patients are listed along with links to patient-specific pages, each patient-specific page corresponding to one of said plurality of patients, at least a portion of said patient-specific pages displaying biometric data, said provider portal also being configured to display said patient-specific pages upon selection by said provider; and
a computational module being configured to record session time said provider spends on each of said patient-specific pages in a given session, and being configured to calculate total time said provider spent on said patient-specific pages for all sessions within said billing period for each of said plurality of patients.

2. The WMS system of claim 1, wherein said computational module is configured to calculate said total time by itemizing time spent on each of said patient-specific pages for a given patient for all sessions within said billing period.

3. The WMS system of claim 1, wherein, for said billing period, a session time is below said minimum unit of time a provider must spend on a single patient, but said total time is above said minimum unit of time.

4. The WMS system of claim 1, wherein said computational module is configured to monitor provider activity while on one of said patient-specific pages, and wherein said computational module is configured to prompt said provider after a predetermined first period of detecting no provider activity while on said patient-specific page to determine if said provider is still viewing said patient-specific page, and if said provider does not respond to said prompt affirmatively, then a timer is stopped.

5. The WMS system of claim 4, wherein a predetermined second period is subtracted from the session time if said provider does not respond affirmatively to said prompt.

6. The WMS system of claim 5, wherein said predetermined first period is not less than said predetermined second period.

7. The WMS system of claim 1, wherein said biometric data includes at least one of the following: spirometer data, blood pressure data, heart rate data, weight, oxygen saturation, blood glucose level, or body temperature.

8. The WMS system of claim 1, wherein said patients are ordered on said general page such that a patient whose biometric data exceeds one or more limits is listed above a patient whose biometric data is within said one or more limits.

9. The WMS system of claim 1, wherein a particular biometric measurement of said portion of said biometric measure data displayed on said general page is emphasized if it exceeds a predetermined limit.

10. The WMS system of claim 1, wherein hovering over one of said links to said patient-specific pages causes said portal to display a patient-specific page depicting a trend graph of said biometric measurement.

11. The WMS system of claim 1, wherein said patient-specific pages relate to services other than monitoring biometric data.

12. The WMS system of claim 11, wherein said services other than monitoring biometric data includes at least one of videoconferencing and logged activities.

13. The WMS system of claim 12, wherein said provider portal comprises a video/phone conferencing patient-specific page which facilitates real-time communication with a patient

14. The WMS system of claim 13, wherein said computational module monitors said time provider is on said video/phone conferencing page for said patient.

15. The WMS system of claim 1, wherein said patient portal is configured to register a patient's computer.

16. The WMS system of claim 15, wherein said computer is linked to one or more devices for obtaining said biometric data.

17. The WMS system of claim 16, wherein said computer is a tablet or a spirometer.

18. The WMS system of claim 16, wherein at least a portion of said one or more devices transmit said biometric data wirelessly.

19. A Wellness Management Services (WMS) method for use in a healthcare system in which a healthcare provider must spend a minimum unit of time on a single patient within a billing period to bill for said time, said WMS method comprising:
displaying a general page in which a plurality of patients are listed along with links to patient-specific pages, each patient-specific page corresponding to one of said plurality of patients, at least a portion of said patient-specific pages displaying biometric data, said provider portal also being configured to display said patient-specific pages upon selection by said provider;
automatically recording session time said provider spends on each of said patient-specific pages in a given session;
automatically calculating total time said provider spends on said patient-specific pages for all sessions within said billing period for each of said plurality of patients; and
providing a record of said total time in a form sufficient to meet health-care documentation requirements for insurance reimbursement.

20. The WMS method of claim 19, wherein, for said billing period, a session time is below said minimum unit of time a provider must spend on a single patient, but said total time is above said minimum unit of time.
